(19) United States
(12) Patent Application Publication
(10) Pub. No.: US 2014/0356897 A1
(43) Pub. Date: Dec. 4, 2014

(54) IN VIVO LABEL-FREE HISTOLOGY BY PHOTOACOUSTIC MICROSCOPY OF CELL NUCLEI

(71) Applicant: The Washington University, St. Louis, MO (US)

(72) Inventors: Lihong Wang, St. Louis, MO (US); Da-Kang Yao, St. Louis, MO (US); Konstantin Maslov, St. Louis, MO (US)

(21) Appl. No.: 14/362,703

(22) PCT Filed: Dec. 7, 2012
(86) PCT No.: PCT/US2012/068403

§ 371 Date: Jun. 4, 2014

Related U.S. Application Data
(60) Provisional application No. 61/568,249, filed on Dec. 8, 2011.

(51) Int. Cl. G01N 33/483; A61B 5/00; C12Q 1/02

(52) U.S. Cl. CPC G01N 33/4833; C12Q 1/02; A61B 5/0095
USPC 435/29; 435/288.7; 600/407

(57) ABSTRACT

The present disclosure is directed to methods of non-invasively imaging cell nuclei. More particularly, the present disclosure is directed to methods of imaging cell nuclei in vivo using ultraviolet photoacoustic microscopy (UV-PAM), in which ultraviolet light is used to excite unlabeled DNA and RNA in cell nuclei to produce photoacoustic waves.

### CROSS-REFERENCE TO RELATED APPLICATIONS

The present disclosure claims priority to U.S. provisional patent application No. 61/568,249, filed on Dec. 8, 2011, which is incorporated herein by reference in its entirety.

### STATEMENT REGARDING FEDERALLY SPONSORED RESEARCH OR DEVELOPMENT

The present disclosure was made with government support under grant number R01 EB000712 awarded by the National Institutes of Health. The government has certain rights in the invention.

### BACKGROUND OF THE DISCLOSURE

The present disclosure relates generally to cell imaging. More particularly, the present disclosure is directed to non-invasively imaging cell nuclei in vivo without staining using ultraviolet photoacoustic microscopy (UV-PAM) to produce photoacoustic waves.

Cell nuclei are organelles containing the DNA genome, in which major cell activities take place, such as DNA replication, RNA synthesis, and ribosome assembly. Since cancer cells lose their control of DNA replication, their nuclei are different from normal nuclei in morphology. For example, two morphological characteristics of the nuclei in cancer cells are their folded shape and enlarged size. These characteristics are considered the hallmarks of cancer and are used by pathologists to determine cancer grade and evaluate prognosis. Therefore, imaging of cell nuclei plays a critical role in cancer diagnosis.

Imaging cell nuclei is conventionally conducted using invasive ex vivo histology, which requires tissue excision and staining (i.e., labeling). Excised tissue goes through a complicated histological process, including tissue processing, embedding, sectioning, and staining, before imaging with microscopy. Methods such as, for example, reflectance confocal microscopy, fluorescence confocal microscopy, and multiphoton microscopy have been used to successfully image cell nuclei without sectioning. However, reflectance confocal microscopy requires staining the cell nuclei using citric acid, acridine orange, or methylene blue, for example. Without staining cell nuclei, reflectance confocal microscopy relies on the difference in refractive indices between cell nuclei and other tissue microstructures for image contrast. Multiphoton microscopy produces in vivo images of cell nuclei by detecting autofluorescence of reduced nicotinamide adenine dinucleotide (NADH), and thus, multiphoton images show cell nuclei in negative contrast as perinuclear fluorescent speckles. Third harmonic light is generated by lipid bodies and collagen in tissue. Third harmonic generation microscopy can employ backscattered third harmonic light to construct images of tissue structure, even though the image contrast is relatively low for cell nuclei. Therefore, imaging of cell nuclei still falls short of a label-free imaging technique with high contrast and spatial resolution.

Photoacoustic microscopy (PAM) is an imaging technique with rich optical contrast and high spatial resolution. In PAM, a pulsed laser beam is focused into biological tissue. Once the light pulse is absorbed by the tissue and converted into heat, thermoelastic expansion of the tissue generates ultrasonic waves, which are detected by a focused transducer. By using light absorbed by the hemoglobin in red blood cells, PAM is capable of imaging the vasculature in various tissues, such as the human skin and the mouse brain. Furthermore, PAM is capable of mapping the total concentration and the oxygen saturation of hemoglobin in single blood vessels by using two or more optical wavelengths in the visible spectral region.

Although methods are available for imaging cell nuclei, these methods involve complicated histological processes before microscopic imaging. Accordingly, there exists a need to develop alternative methods for imaging cell nuclei for cancer diagnosis and prognosis.

### SUMMARY OF THE DISCLOSURE

The present disclosure relates generally to cell imaging. More particularly, the present disclosure is directed to non-invasively imaging cell nuclei in vivo without staining using ultraviolet photoacoustic microscopy (UV-PAM) to produce photoacoustic waves.

In one aspect, the present disclosure is directed to an ultraviolet photoacoustic imaging apparatus. The ultraviolet photoacoustic imaging apparatus comprises: a focusing assembly configured to receive ultraviolet light and to focus the ultraviolet light on a biological sample; at least one transducer configured to receive photoacoustic waves emitted by the biological sample in response to the ultraviolet light, said at least one transducer positioned such that said at least one transducer and said focusing assembly are coaxial; and a processor configured to record and process the received photoacoustic waves.

In another aspect, the present disclosure is directed to a method for imaging a cell nucleus in a biological sample. The method comprises: exposing the biological sample to ultraviolet light, wherein the ultraviolet light is focused on an area of the biological sample using a focusing assembly; transforming optical energy absorbed by the area of the biological sample in response to the ultraviolet light into a photoacoustic wave; detecting the photoacoustic wave using at least one transducer positioned such that the transducer and the focusing assembly are coaxial; and creating an image of the area of the biological sample using a processor, the image being based on a signal generated by the transducer and being representative of the photoacoustic wave.

In another aspect, the present disclosure is directed to a method for imaging an area of a biological sample in vivo. The method comprises: exposing an area of the biological sample to ultraviolet light, wherein the ultraviolet light is focused on the area of the biological sample using a focusing assembly; transforming optical energy absorbed by the area of the biological sample in response to the ultraviolet light into a photoacoustic wave; detecting the photoacoustic wave using at least one transducer positioned such that the transducer and the focusing assembly are coaxial; and creating an image of the area of the biological sample using a processor, the image being based on a signal generated by the transducer and being representative of the photoacoustic wave.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a schematic of an exemplary UV-PAM imaging apparatus of the present disclosure.

FIG. 2 is a schematic of an exemplary UV-PAM imaging apparatus of the present disclosure.

FIG. 3A is a UV-PAM image of the cross section of the small intestine as described in Example 2.

FIG. 3B is a non-color (black-and-white) histological image of the section stained with hematoxylin and eosin as described in Example 2.

FIG. 3C is a close-up of the area enclosed by dashed lines in FIG. 3A as described in Example 2.

FIG. 3D shows an optical micrograph of the cell nuclei in FIG. 3B as described in Example 2.

FIG. 4A is an image of nuclei of the epithelial cells in the mouse lip as described in Example 3.

FIG. 4B is an image of nuclei of the epithelial cells in the mouse small intestine as described in Example 3.

FIG. 5 is a three-dimensional (3D) UV-PAM image of the cell nuclei in the mouse ear skin at different depths as described in Example 4.

FIG. 6 shows in vivo en face photoacoustic images of the skin of mouse ears in the form of maximum amplitude projection (MAP) acquired using wavelengths of 240, 245, 248, 250, 251, 252, 255, 260, 266, 270, 275, and 280 nm as described in Example 5.

FIG. 7A is in vivo en face distribution of cell nuclei in the skin of mouse ears as described in Example 6.

FIG. 7B is a histogram of the nuclear diameter as described in Example 6.

FIG. 7C is a histogram of the internuclear distance as described in Example 6.

FIG. 8A shows a plot of SNR (mean±SD) of nuclear images versus wavelength as described in Example 7.

FIG. 8B shows a plot of CNR (mean±SD) of nuclear images versus wavelength as described in Example 7.

FIG. 8C shows the absorption spectra of thymus DNA and the protein glutamate dehydrogenase as described in Example 7.

FIG. 8D shows the ratio of DNA to protein, defined as the ratio of the absorption coefficient of thymus DNA to that of glutamate dehydrogenase as described in Example 7.

FIG. 9A shows in vivo MAP photoacoustic images of cell nuclei at pulse energies of 10, 3, and 2 nJ as described in Example 8.

FIG. 9B shows the CNR (mean±SD) of the nuclear images for each pulse energy as described in Example 8.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

While the making and using of various embodiments of the present disclosure are discussed in detail below, it should be appreciated that the presently described embodiments provide many applicable inventive concepts that may be embodied in a wide variety of contexts. The embodiments discussed herein are merely illustrative of exemplary ways to make and use embodiments of the disclosure and do not delimit the scope of the present disclosure.

To facilitate the understanding of the presently described embodiments, a number of terms are defined below. Terms defined herein have meanings as commonly understood by a person of ordinary skill in the areas relevant to aspects of the disclosure. Terms such as “a,” “an,” “the,” and “said” are not intended to refer to only a singular entity, but include the general class of which a specific example may be used for illustration and are intended to mean that there are one or more of the elements. The terms “comprising,” “including,” and “having” are intended to be inclusive and mean that there may be additional elements other than the listed elements.

The order of execution or performance of the operations in embodiments of the disclosure illustrated and described herein is not essential, unless otherwise specified. That is, the operations may be performed in any order, unless otherwise specified, and embodiments of the present disclosure may include additional or fewer operations than those disclosed herein. For example, it is contemplated that executing or performing a particular operation before, contemporaneously with, or after another operation is within the scope of aspects of the present disclosure.

The terms used herein follow the definitions recommended by the Optical Society of America (OCIS codes).

As used herein, the term “ultraviolet photoacoustic microscopy” (or “UV-PAM”) refers to a photoacoustic imaging technology that detects pressure waves generated by ultraviolet light absorption in the volume of a material such as, for example, a biological sample, and propagated to the surface of the material. Ultraviolet photoacoustic microscopy also refers to a method for obtaining images of the optical contrast of a material by detecting photoacoustic and/or pressure waves traveling from an area of the biological sample under investigation. Moreover, ultraviolet photoacoustic microscopy refers to the detection of the pressure waves that may still be within the biological sample. Without being bound by theory, DNA (deoxyribonucleic acids) and RNA (ribonucleic acids) strongly absorb particular wavelength UV light. The absorbed light is then converted into heat in the nucleus of a cell. Thermoelastic expansion as a result of the heat generates photoacoustic waves.

As used herein, the term “photoacoustic tomography” refers to a photoacoustic imaging technology that detects photoacoustic and/or pressure waves generated by light absorption in the volume of a material such as, for example, a biological sample, and propagated to the surface of the material.

As used herein, the term “time-resolved detection” refers to the recording of the time history of a pressure wave with a temporal resolution sufficient to reconstruct a pressure wave profile.

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In accordance with the present disclosure, devices and methods have been discovered that allow for imaging cell nuclei. More particularly, the present disclosure is directed to methods for non-invasively imaging cell nuclei in vivo or ex vivo without staining or preparing thin slides using ultraviolet photoacoustic microscopy (UV-PAM) to produce photoacoustic waves.

In UV-PAM, ultraviolet (UV) light is used in place of visible light. DNA and RNA—two major compounds in cell nuclei—strongly absorb UV light. In contrast, the UV absorption of protein and lipids is weaker than that of DNA and RNA.

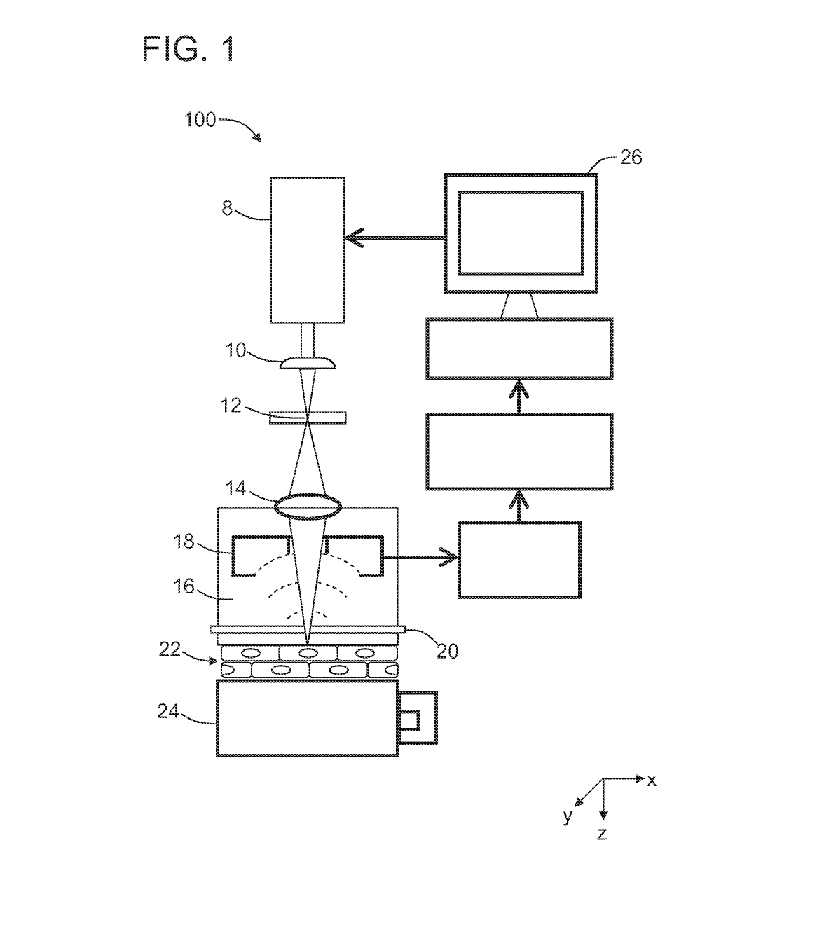

In one aspect, the present disclosure is directed to an ultraviolet photoacoustic imaging apparatus as shown in FIG. 1. The ultraviolet photoacoustic imaging apparatus 100 generally includes a focusing assembly 10 configured to receive ultraviolet light (UV) from a UV light emitting device 8 (e.g., UV laser) and to focus the UV light on a biological sample 22. Typically, the UV light is emitted from the UV light source such as a UV laser in light pulses.

The wavelength of UV light may range from about 210 nm to about 320 nm. In particular, suitable ultraviolet light wavelengths may be from about 240 nm to about 280 nm, including from about 245 nm to about 275 nm. More particularly, the UV light wavelengths may be from about 248 nm to about 265 nm. In one specific embodiment, the UV light source 8 emits UV light at a wavelength of about 250 nm.

Any equipment suitable for emitting UV light for microscopy may be useful in the present disclosure. For example, UV light may be emitted using a QL266-010-O Qswitched UV laser, commercially available from Crystalaser (Reno, Nev.), pumped by a neodymium-doped yttrium lithium fluoride (Nd:YLF). The UV light may be, for example, intensity modulated UV light and pulsed UV light. Suitable UV light pulse duration may be from about 1 ns to about 20 ns, including from about 1 ns to about 10 ns.

In one particularly suitable embodiment, the focusing assembly 100 is a condenser lens 10 positioned over a diaphragm (pinhole) 12 for spatial filtering. Suitable diaphragm diameters may be from about 10 μm to about 100 μm. In one exemplary embodiment, the UV light is spatially filtered by a 25-μm diameter diaphragm (for example, the 910PH-25, commercially available from Newport Corporation (Irvine, Calif.)). In some embodiments, the UV light coming out of the diaphragm 12 may then further be focused by a water-immersion microscope objective lens 14 (for example, an LB4280 lens, commercially available from Thorlabs, Inc. (Newton, N.J.)) into a water tank 16 to focus the UV light into a nearly diffraction-limited point.

In some embodiments, the focusing assembly may comprise an optical assembly of lenses and mirrors configured to focus the UV light on the biological sample in such a way that a focal point of said focusing assembly coincides with a focal point of a transducer as described herein.

The focusing assembly may be positioned on an XYZ translation stage 24 to perform raster scanning along a surface of the biological sample with simultaneous adjustment of an axial position of said apparatus to compensate for a curvature of the surface of the biological sample. The focusing assembly is placed on an XYZ translation stage to perform raster scanning along the biological sample surface with simultaneous adjustment of the sensor's axial position to compensate for the curvature of the biological sample surface.

The recorded pressure-wave time histories are displayed by a computer processor as described herein versus the focusing assembly position to construct a three dimensional image of the distribution of the optical contrast within the biological sample, i.e., a three dimensional tomographic image of the biological sample. Other embodiments may use different methods of image formation such as, for example, circular scanning, sector scanning, optical scanning, electronic focusing a transducer array, array-based image reconstruction, and combinations thereof.

In one embodiment, the focusing assembly comprises an oscillating mirror configured to scan an optical focus of a larger focal area as compared to a focal area of the transducer.

As described briefly above, at least one transducer 18 is configured to receive photoacoustic waves emitted by the biological sample 22 in response to the UV light. In one embodiment, the transducer 18 is an ultrasonic transducer. More particularly, in an exemplary embodiment, a ring-shaped focused ultrasonic transducer (center frequency, 50 MHz; focal length, 7 mm) is used as the ultrasonic transducer to receive the photoacoustic waves. As used herein, “focused ultrasonic transducer” refers to a curved ultrasonic transducer with a hemispherical surface or a planar ultrasonic transducer with an acoustic lens attached or an electronically focused ultrasonic array transducer.

In one exemplary embodiment, as shown in FIG. 1, the UV light passes through the ultrasonic transducer 18 and penetrates an imaging window membrane 20 before the light focuses on the biological sample 22 to be imaged. In one embodiment, the membrane of the imaging window 20 is a 25 μm-thick polyethylene membrane. The polyethylene membrane seals the bottom of the water tank to form an imaging window while maintaining acoustic coupling. Suitable UV laser pulse energy may range from about 1 nJ to about 100 nJ. In particular, suitable UV laser pulse energy behind the membrane may be about 1 nJ to about 35 nJ. More particularly, the UV laser pulse energy may be from about 1 nJ to about 20 nJ, including from about 1 nJ to about 10 nJ, and including from about 2 nJ to about 10 nJ.

Suitably, the transducer is positioned such that the transducer and the focusing assembly are in coaxial and confocal relationship. During operation, the biological sample 22 is mounted on XYZ translation scanning stage 24 with a minimal scan step size (for example, a step size of approximately 0.31 μm). Photoacoustic waves generated in the sample 22 by the UV light are recorded and analyzed by a computer processor 26 to form a three-dimensional image. The shape and dimensions of the optical-contrast structures are generally determined from the temporal profile of the photoacoustic waves and the position of the focusing assembly. Conventionally, a raster scan by the focusing assembly may be used to form a three-dimensional image. For example, time-resolved photoacoustic signals may be detected by the transducer 18 during raster scanning to reconstruct tomographic images, which may be rendered in various forms such as, for example, cross-sectional images and maximum amplitude projection (MAP) images. Additionally, or alternatively, however, a transducer array may be used to reduce the time of scanning and the total light exposure.

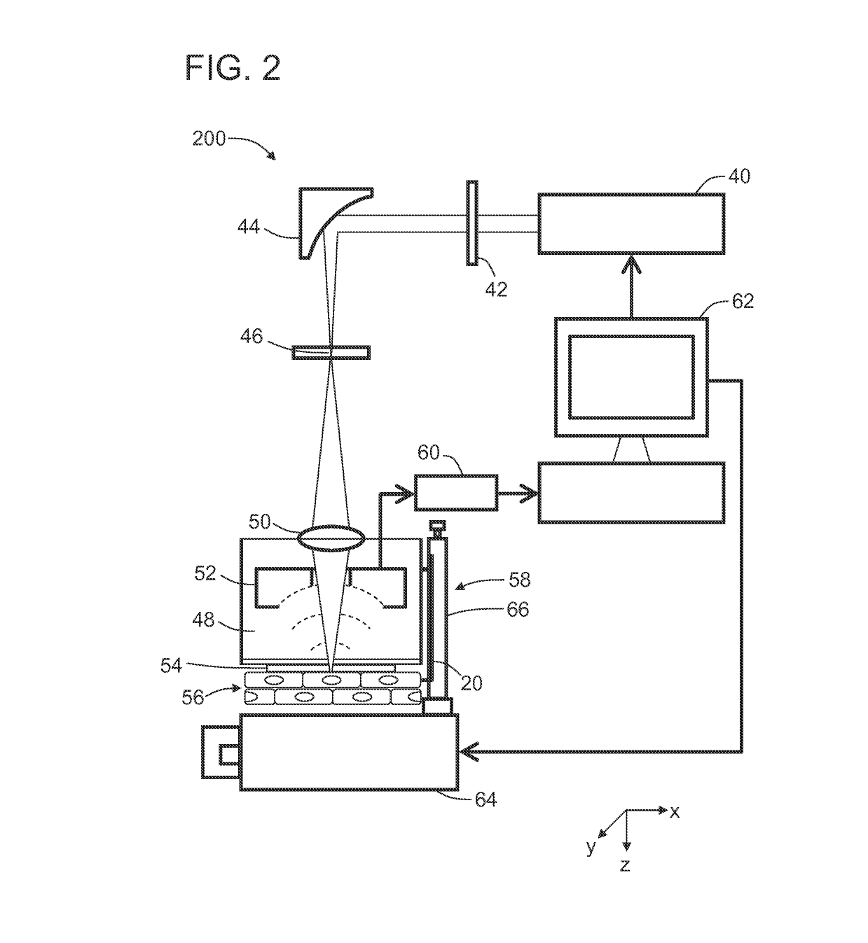

In another exemplary embodiment, as shown in FIG. 2, a UV-PAM apparatus 200 is equipped with an UV light emitting device 40 such as, for example, an optical parametric oscillator (OPO) UV laser system (for example, a NT242-SH, commercially available from Altos Photonics (Bozeman, Mont.)) that provides a UV wavelength tuning range from 210 to 2300 nm as compared to a fixed wavelength UV laser. A pulsed UV light with a pulse width of 5 ns was emitted from the UV light emitting device 40 at a repetition rate of 1 kHz. Other suitable pulse widths may be, for example, from about 1 ns to about 10 ns. Other suitable repetition rates may be, for example, from about 1 kHz to about 500 kHz. After being attenuated by a neutral density (ND) filter 42 (for example, an NDC-50C-4M, commercially available from Thorlabs, Inc. (Newton, N.J.)), the UV light is focused by a 100-mm-focal-length off-axis parabolic mirror 44 (for example, a 50338AL, commercially available from Newport Corporation (Irvine, Calif.)), and then spatially filtered by a 25 μm diameter pinhole 46 (for example, a 910PH-25, commercially available from Newport Corporation (Irvine, Calif.)). The UV light is refocused into a water tank 48 by a 0.1 NA objective lens 50 (for example, an LA4280 lens, commercially available from Thorlabs, Inc. (Newton, N.J.)), passes through a focused ring ultrasonic transducer 52 (50 MHz central frequency, 7 mm focal length), and penetrates a 25 μm thick polyethylene membrane 54 before focusing along the z axis to an biological sample (e.g., tissue) 56. The ultrasonic transducer 52 is coaxially aligned with the objective lens 50 to a common focus. The polyethylene membrane 54 forms an optical window on the bottom of the water tank 48 and maintains acoustic coupling. The UV light pulse energy behind the polyethylene membrane 54 is measured by a digital power meter (for example, a PM100D, commercially available from Thorlabs, Inc. (Newton, N.J.)) with a silicon photodiode sensor (for example, a S120VC, commercially available from Thorlabs, Inc. (Newton, N.J.)). The water tank 48 and the biological sample 56 (e.g., tissue) are mounted on a translation stage 58 that has a motorized scanning stage 64 (commercially available from Micos USA (Irvine, Calif.)) and a manual vertical translator 66 (for example, a PT1, commercially available from Thorlabs, Inc. (Newton, N.J.)) for manual focusing. The UV-PAM apparatus 200 may be equipped with a scanning stage 58 to perform raster scanning in the horizontal plane (x-y plane). Raster scanning may be performed at any desired step size. A particularly suitable step size may be, for example 0.62 μm. Other suitable step sizes may be, for example, from about 0.1 μm to about 2 μm. While the scanning stage 58 performs raster scanning in the horizontal plane (x-y plane), photoacoustic signals are detected by the ultrasonic transducer 52, amplified by an amplifier 60 (for example, a ZFL-500LN, commercially available from Mini-Circuits (Branson, Mo.)), and collected by a computer processor 62 through a 12-bit, 200 MHz digitizer (not shown; for example, a NI PCI-5124, commercially available from National Instruments (Austin, Tex.)). Tomographic images are formed from the amplitude envelopes of the time-resolved photoacoustic signals.

Each UV light pulse produces a time-resolved photoacoustic signal. Hilbert transformation of the signal produces its amplitude envelope along the z-axis. A collection of the envelopes along the x-axis produces a cross-sectional image in the x-z plane, a B-scan image. Further scanning along the y-axis produces three-dimensional images. Projection of the maximal amplitude of each envelope to the scanning plane (x-y plane) produces a maximum amplitude projection (MAP) image.

In another aspect, the present disclosure is directed to a method for imaging a cell nucleus in a biological sample using the ultraviolet photoacoustic imaging apparatus described herein. The method includes exposing the biological sample to ultraviolet light, wherein the ultraviolet light is focused on an area of the biological sample using the focusing assembly; transforming optical energy absorbed by the biological sample in response to the ultraviolet light into a photoacoustic wave; detecting the photoacoustic wave using a transducer, such as, for example, an ultrasonic transducer, positioned such that the transducer and the focusing assembly are coaxial; and creating an image of the area of the biological sample using a processor, the image being based on a signal generated by the transducer and being representative of the photoacoustic wave.

The biological sample may be, for example, any organism, organ, tissue, and cell having a nucleus within which genetic material (e.g., DNA and RNA) is carried. For example, the sample may be a eukaryotic organism. The eukaryotic organism may be, for example, a vertebrate, an invertebrate, a plant, a fungus, or a single-cellular organism.

In one particular embodiment, the present disclosure is directed to a method for non-invasively imaging a cell nucleus in vivo. The term “in vivo” is used herein according to its ordinary meaning as understood by one skilled in the art to refer to UV-PAM performed with the biological sample in a living organism in its normal, intact state. The term also refers to conditions requiring, for example, anesthetizing the living organism.

While described herein as imaging a cell nucleus, it should be understood that other areas of a biological sample can be imaged using the UV-PAM imaging apparatus of the present disclosure at proper wavelength. Other areas that may be imaged may be, for example, the cytoplasm and nucleolus.

Alternatively, the biological sample may be ex vivo, in vitro, and combinations thereof. The term “ex vivo” is used herein according to its ordinary meaning as understood by one skilled in the art to refer to UV-PAM performed with the biological sample in an artificial environment outside the organism with the minimum alteration of natural conditions. The biological sample ex vivo may be, for example, an intact organ removed from an organism, but not further processed (for example, sectioned and/or stained). The biological sample ex vivo may also be, for example, an organ that is removed from the organism and cut to expose surfaces or structures internal to the organ as it is removed from the organism. The term “in vitro” is used herein according to its ordinary meaning as understood by one skilled in the art to refer to UV-PAM performed with the biological sample that has been isolated from its usual biological context to permit analysis of the biological tissue than cannot be done with the whole organism or organ. The biological sample in vitro may be, for example, an organ that is removed from an organism that is further processed such as, for example, embedded and/or sectioned as would be done for histological purposes, but not stained or otherwise processed (e.g., staining).

In another aspect of the method, the ex vivo biological sample may further be histologically processed. For example, cell nuclei may be imaged ex vivo using UV-PAM followed by histological processing (e.g., embedded, sectioned, and/or stained) for imaging cell nuclei using conventional histological imaging. While staining of the biological sample is not required for UV-PAM imaging of the biological sample, staining of the biological sample may be performed as described herein.

Suitable wavelengths may be, for example, between about 210 nm and about 310 nm. More suitably, wavelengths may be, for example, between about 240 nm and about 280 nm. Particularly suitable wavelengths may be about 245 nm to about 275 nm.

The disclosure will be more fully understood upon consideration of the following non-limiting Examples.

### EXAMPLES

### Example 1

In this Example, spatial resolution of the UV-PAM imaging apparatus was measured.

Specifically, single submicrometer beads were imaged to measure the spatial resolution of the UV-PAM imaging apparatus. The beads were black polystyrene microspheres 0.2 μm in diameter (Polysciences), immobilized by adsorption on a quartz slide (Chemglass Life Sciences). Bead images were acquired by scanning with a 0.31 μm step size. The lateral FWHM was measured by fitting the Airy pattern to the amplitude profiles of the horizontal cross-sectional images of the single beads and the axial FWHM was measured by Gaussian fitting to the axial amplitude profiles of bead images. By fitting the images of 15 beads, the lateral FWHM was found to be 0.70±0.04 um (mean±standard error) and the axial FWHM to be 28.5±0.8 μm.

### Example 2

In this Example, images of cross sections of mouse small intestine obtained using UV-PAM were compared with images of the same tissue after hematoxylin and eosin histological staining.

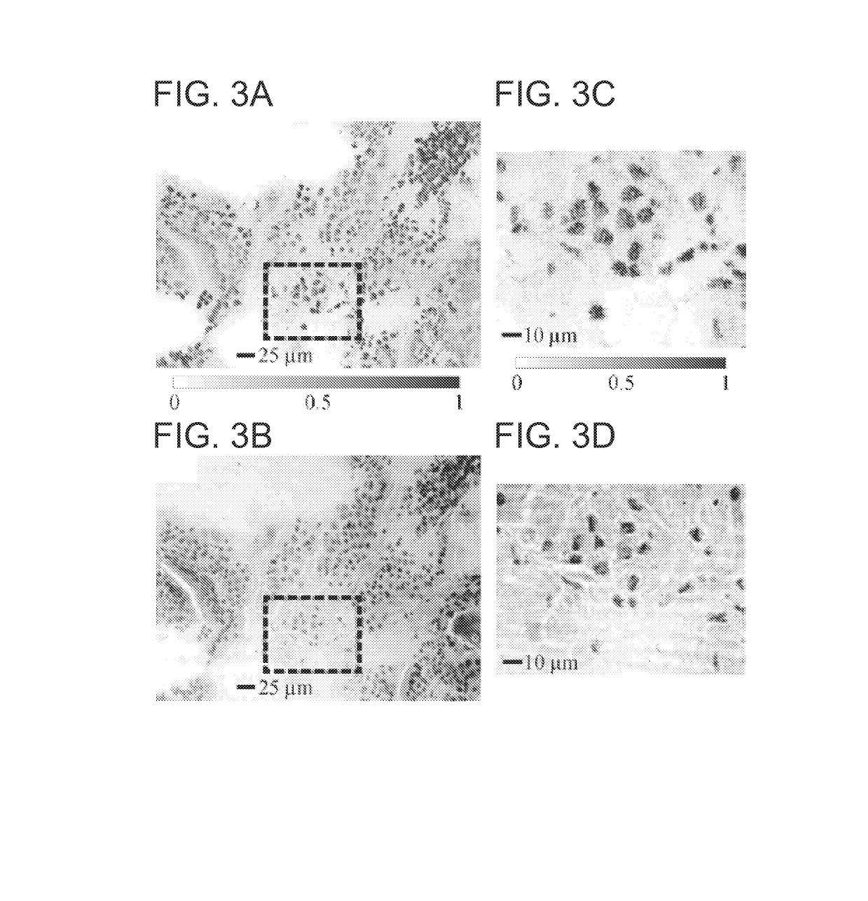

Specifically, small intestine was excised from a sacrificed Swiss Webster mouse (Harlan Laboratories), and cut into 6-μm-thick cross sections by a cryostat (CM1850; Leica Microsystems). A UV-PAM image of the cross section of the small intestine was acquired by scanning the tissue section with a 0.62 μm step size (FIG. 3A). After the scanning, the section slide was stained by hematoxylin and eosin (Sigma-Aldrich). Hematoxylin stains the cell nucleus blue, and eosin stains the cytoplasm pink. Next, optical micrographs of the intestine section were obtained using a microscope with a 20× objective (0.45 NA, Nikon).

FIG. 3B shows the cell nuclei as dark spots, as expected in histology (in a color image of a hematoxylin-eosin stained tissue, the cell nuclei appear as dark-blue spots). A comparison of FIG. 3A with FIG. 3B shows that each dark spot in the UV-PAM image shown in FIG. 3A corresponds to a nucleus of a cell stained by hematoxylin in FIG. 3B. These results confirmed that cell nuclei (dark spots of FIG. 3A) were imaged by UV-PAM. Because UV-PAM possesses background-free detection, the cell nuclei are shown in high positive contrast owing to both the strong UV absorption of DNA and RNA and the weak absorption of cytoplasm. The signal-to-noise ratio was measured to be as high as 52 dB. FIG. 3A further demonstrated the same spatial distribution of cell nuclei as shown in FIG. 3B. As shown in a close-up in FIG. 3C, UV-PAM was able to resolve the shape and size of the cell nuclei. FIG. 3D shows an optical micrograph of the same cell nuclei in the hematoxylin-eosin stained tissue obtained with a 60× oil-immersion objective (1.4 NA, Nikon). The shape and size of the cell nuclei in FIG. 3C are consistent with those in FIG. 3D. These results demonstrate the capability of UV-PAM for label-free histology.

### Example 3

In this Example, cell nuclei in the epithelium of a mouse lip and in the intestinal villi of a mouse small intestine were imaged using UV-PAM ex vivo.

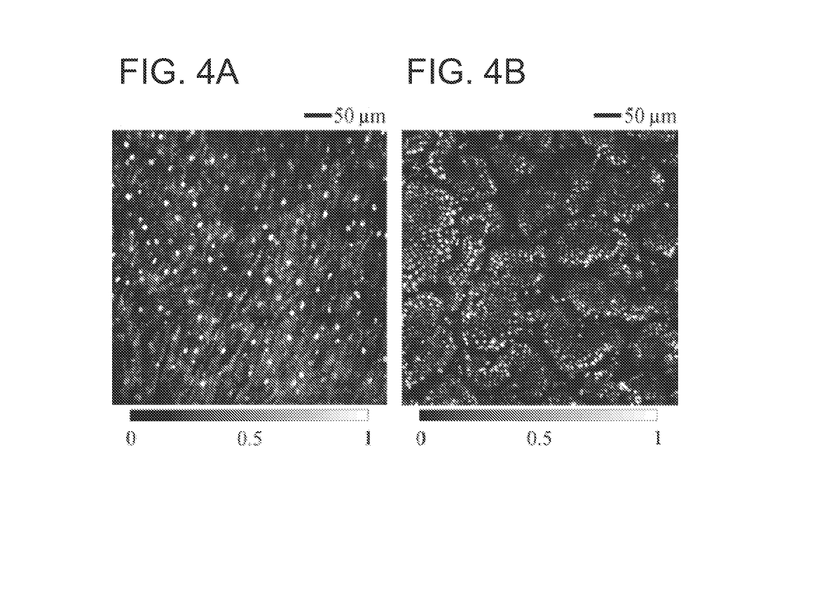

Fresh specimens were taken from adult Swiss Webster mice and immersed in phosphate buffer solution (PBS, Sigma-Aldrich). After the small intestine was cut longitudinally and unfolded into a sheet, the specimens were mounted on the scanning stage with their inner surfaces in contact with the image window through PBS. The nuclei of the epithelial cells in the mouse lip were imaged by scanning with a 1.25 μm step size for 2.6 min. As shown in FIG. 4A, the image shows a relatively homogeneous distribution of cell nuclei, each approximately 6 μm diameter. The distance between the centers of neighboring cell nuclei ranged from 16 μm to 39 μm, suggesting that the stratified squamous epithelium on the lip was composed of cells with a lateral size of the same range. The nuclei of the epithelial cells on the mouse small intestine were imaged by scanning with a 0.62 μm step size for 7.4 min. In contrast to the stratified squamous epithelium on the lip, the image in FIG. 4B shows the tight arrangement of simple columnar epithelial cells in intestinal villi. The nuclear diameter was ˜3 μm, and the cell width was ˜6 μm. These results demonstrate that UV-PAM is capable of imaging cell nuclei in nonsectioned tissues.

### Example 4

In this Example, UV-PAM was used to image the cell nuclei in vivo in the ear skin of an athymic nude mouse.

An athymic nude mouse (Harlan Laboratories, Indianapolis, Ind.) was anesthetized using isoflurane and held using a self-constructed stereotaxic imaging stage. A UV-PAM system as illustrated in FIG. 2 and described herein was used.

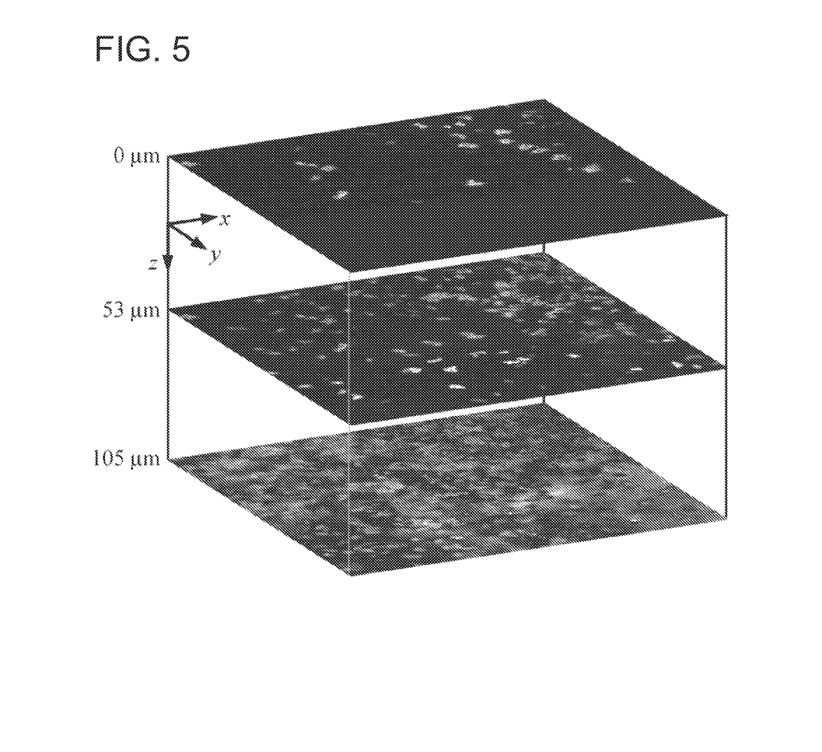

After in vivo by scanning with a 0.62 μm step size, a three-dimensional (3D) image of the cell nuclei in the mouse ear skin was acquired (FIG. 5). Relative to the first slice in FIG. 5, the second and third slices were 53 and 105 μm deep inside the mouse ear, respectively, suggesting that the penetration depth of UV-PAM is greater than 100 μm for in vivo imaging. Cell density varied with the depth: 45 cell nuclei were visualized in the first slice and 159 cell nuclei were visualized in the second slice. Although the lateral resolution of UV-PAM was expected to decrease with penetration depth, 76 cell nuclei were identified in the third slice (at a depth of 105 μm). These results demonstrate that UV-PAM is capable of 3D, non-invasive, in vivo imaging of cell nuclei without staining.

### Example 5

In this Example, UV-PAM was used to image the cell nuclei in mouse ear skin in vivo at various wavelengths using an UV-PAM system with a UV light source having tunable laser.

Four female athymic nude mice were purchased from Harlan Laboratories (Indianapolis, Ind.). The mice were anesthetized with 1% isoflurane (Butler Animal Health Supply, Dublin, Ohio) delivered in pure oxygen (Airgas, St. Louis, Mo.) at a flow rate of 0.5 L/min. Before imaging, the mouse ear skin was gently washed three times using distilled water. After imaging and the inhalational anesthesia was stopped, the mice recovered in ten minutes.

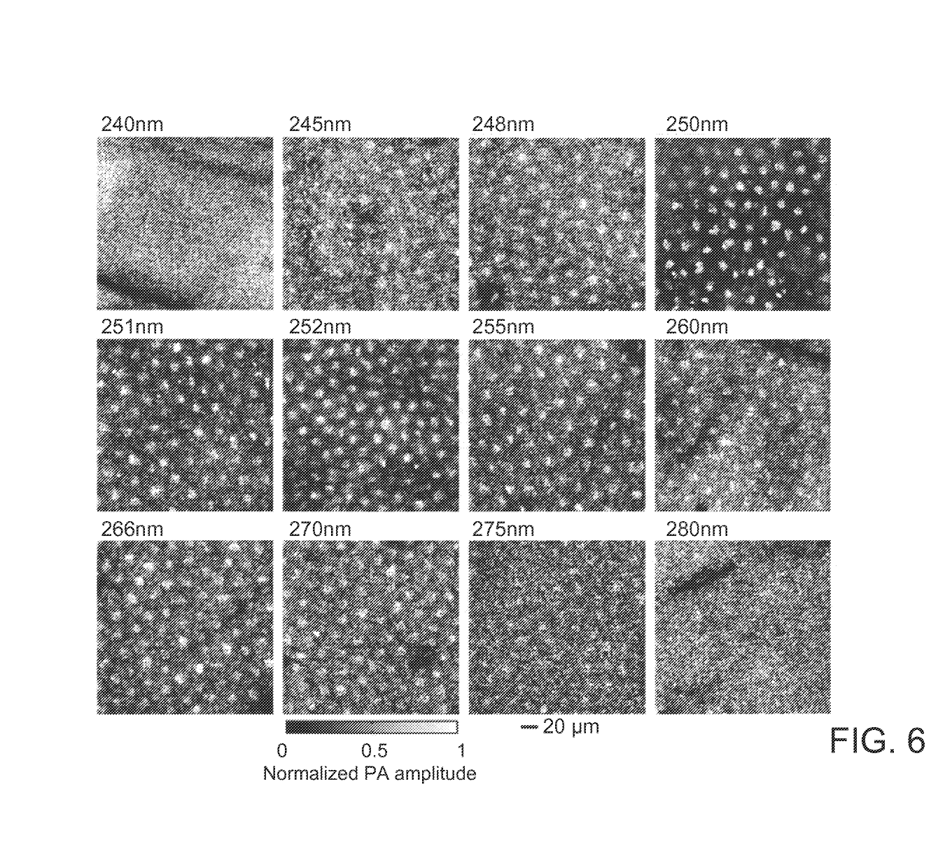

A UV-PAM apparatus was equipped with an OPO UV laser system (NT242-SH, Altos Photonics, Bozeman, Mont.) that provides a UV wavelength tuning range from 210 to 2300 nm. An anesthetized mouse was held by a custom-made stereotaxic imaging stage. After the imaging stage was mounted on the translational stage, a mouse ear was flat placed on a plastic plate immobilized on the imaging stage, and then the image window was lowered to be in contact with a film of distilled water on the mouse ear. During the preview scanning, the translational stage was adjusted upward until a clear B-scan image was observed. Then, the ear skin was scanned with laser pulse energy of 20 nJ at UV wavelengths ranging from 220 to 310 nm. The experiments were repeated twice at wavelengths of 245, 248, 250, 251, 252, 255, 260, 266, 270, and 275 nm. Both experiments yielded identifiable images of cell nuclei. However, for wavelengths of 220, 230, 240, 280, 290, 300, and 310 nm, imaging experiments were repeated two to five times, but did not obtain any identifiable image of cell nuclei (data not shown). Images of mouse ear skin acquired at wavelengths between 240 and 280 nm are shown in FIG. 6. All of these images show that various wavelengths ranging from 245 to 275 nm may be used for in vivo photoacoustic imaging of unstained cell nuclei.

### Example 6

In this Example, UV-PAM was used to examine the distributions of the diameters of cell nuclei and the distance between the centers of neighboring cell nuclei, or the internuclear distance, in the mouse ear skin.

To measure the diameter of a single cell nucleus, a square window with 31×31 pixels was selected to enclose the nucleus image, with the window center close to the nucleus center. Iterative fitting within the square window to Eq. 1

$$I ( x , y ) = A erf ( \frac{R - \sqrt{{( x - {x}_{0} )}^{2} + {( y - {y}_{0} )}^{2}}}{C} ) + B$$

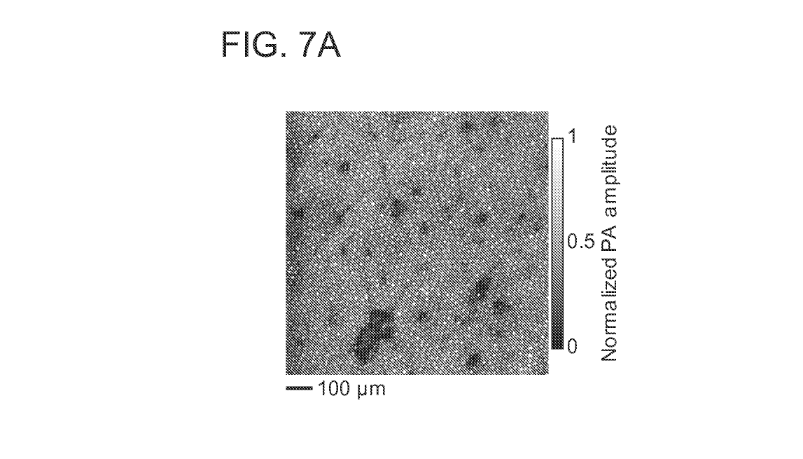

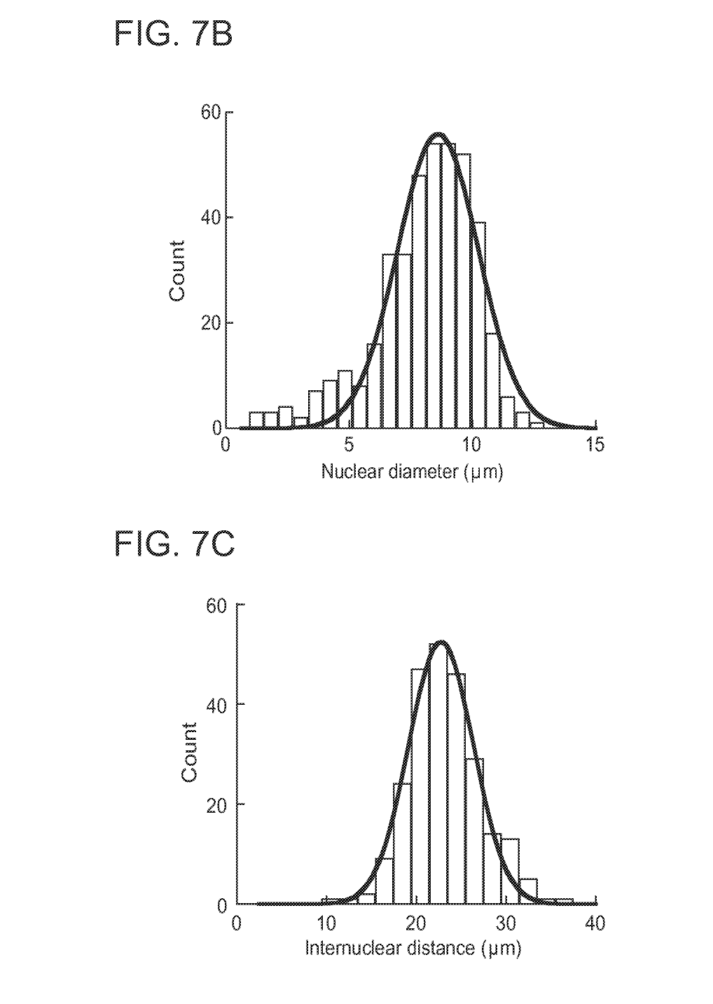

produced both the radius and the center coordinates of the cell nucleus. In Eq. 1, x₀ and y₀ are coordinates of the center of the cell nucleus; R is the radius of the nucleus; A and B are the relative intensity and the background intensity of the image, respectively; and C is a constant. The six parameters, x₀, y₀, R, A, B, and C, are determined by nonlinear least-squares fitting. After fitting 404 cell nuclei from a MAP image acquired at 250 nm with UV laser pulse energy of 20 nJ in FIG. 7A, a histogram of the nuclear diameter of keratinocytes in the mouse skin was obtained as shown in FIG. 7B. After fitting a Gaussian function to the histogram, the nuclear diameter was determined to be 8.6±1.6 μm (mean±standard deviation (SD)) for keratinocytes in the mouse skin. Among the aforementioned 404 cell nuclei, 245 pairs of neighboring cell nuclei were chosen to calculate the internuclear distance. FIG. 7C is a histogram of the internuclear distance of keratinocytes in the mouse skin. After fitting a Gaussian function to the histogram, the internuclear distance was determined to be 22.7±3.6 μm (mean±SD).

### Example 7

In this Example, the signal-to-noise ratio (SNR) and the contrast-to-noise ratio (CNR) of the photoacoustic images of cell nuclei was examined as functions of the optical wavelength.

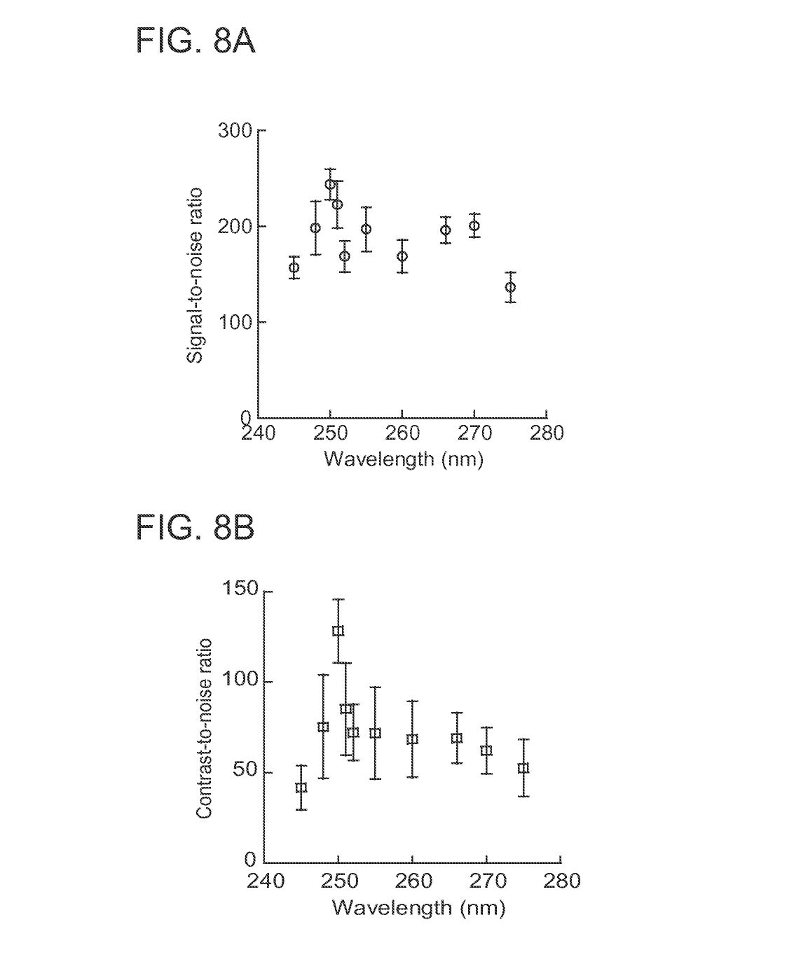

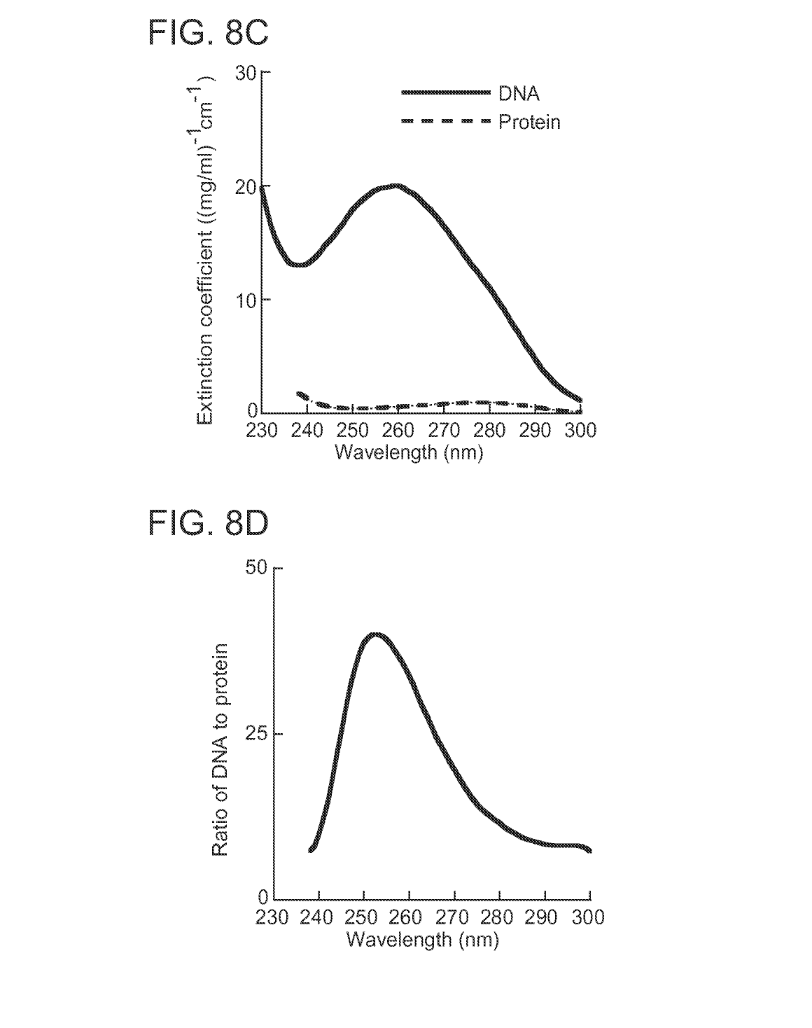

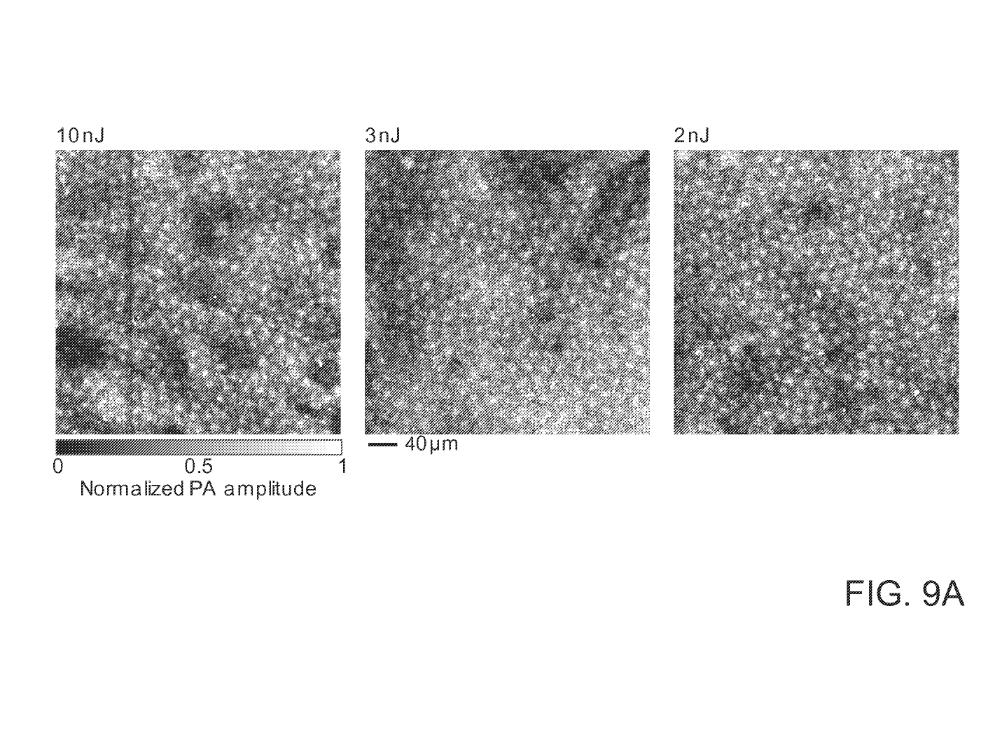

All the images were acquired with UV laser pulse energy of 20 nJ. The mean intensity of the image of a single cell nucleus was obtained by averaging the amplitude within a square window of 10×10 pixels in the image of the cell nucleus. The diameter of cell nuclei is ˜8.6 μm, approximately 14 pixels. Hence, if the cell nucleus image is assumed as a solid circle, a window of 10×10 pixels is an inscribed square in the circle. The distance between the centers of neighboring nuclei is ˜22.7 μm, approximately 36 pixels. Thus, a square window of 30×30 pixels concentric with a single nuclear image does not cover neighboring nuclei, and a square window of 20×20 pixels concentric with the former window totally covers the single nuclear image. Inside the larger window but outside the smaller one, the mean intensity of the background image surrounding the nuclear image was calculated. In order to measure image noise, the mouse ear was scanned using zero laser output in each experiment. A dummy photoacoustic image was formed with zero laser output and was used to compute the standard deviation of image intensity. For each wavelength between 245 and 275 nm, the SNR and CNR of the MAP images of 25 cell nuclei was calculated. After averaging, the dependence of image SNR on wavelength, as shown in FIG. 8A, and the dependence of CNR on wavelength, as shown in FIG. 8B, were obtained. Both SNR and CNR significantly changed with the wavelength, and each reached its maximum at a wavelength of 250 nm. Although application of a 266 nm wavelength generated in vivo photoacoustic images of cell nuclei with high image contrast, the CNR at 250 nm was 1.9 times greater than that at 266 nm, and the SNR at 250 nm was 1.2 times higher than that at 266 nm.

FIGS. 8C and 8D show plots of CNR (mean±SD) of nuclear images and ratio of DNA:protein versus wavelength. FIG. 8C shows a plot of the typical absorption spectra of DNA and protein using thymus DNA and glutamate dehydrogenase. The absorption spectrum of DNA was adapted from Kunitz (1950) and Sambrook et al. (2001), and that of protein from Olson et al. (1952) and Gill et al. (1989). The mean was calculated from 25 nuclei. FIG. 8D shows a plot of the ratio of DNA to protein versus wavelength, defined as the ratio of the absorption coefficient of thymus DNA to that of glutamate dehydrogenase.

### Example 8

In this Example, the in vivo imaging experiment using 250 nm UV light was repeated using a lower UV laser pulse energy.

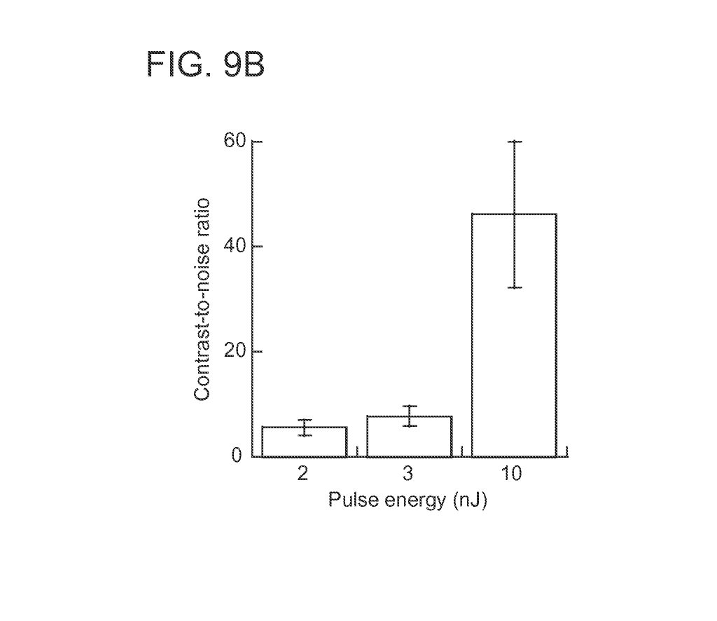

Instead of 20 nJ, the UV laser pulse energy was set to 10, 3, and 2 nJ, yielding three in vivo MAP images of cell nuclei in the mouse ear skin as shown in FIG. 9A. The photoacoustic images showed the regular distribution of cell nuclei in the skin and the nuclear size of individual keratinocytes, even though the laser pulse energy was as low as 2 nJ. FIG. 9B shows the average CNR of the nuclear images at pulse energies of 2, 3, and 10 nJ. The average CNR was 46.1 at 10 nJ, 7.7 at 3 nJ, and 5.5 at 2 nJ. Therefore, the pulse energy limit is less than 2 nJ for in vivo imaging of cell nuclei.

The results presented herein demonstrate that UV-PAM is a novel method that is useful for imaging cell nuclei ex vivo and in vivo. UV-PAM uses optical excitation of DNA and RNA to image cell nuclei. UV-PAM is a non-invasive and label-free method for acquiring cell nuclear images in vivo with strong optical contrast and high spatial resolution. UV-PAM images of unstained cell nuclei match optical micrographs of stained cell nuclei allowing for determination of nuclear shape, size, and distribution. Therefore, UV-PAM has broad applications in cancer studies and diagnosis.

What is claimed is:

1. An ultraviolet photoacoustic imaging apparatus comprising:
a focusing assembly configured to receive ultraviolet light and to focus the ultraviolet light on a biological sample;
at least one transducer configured to receive photoacoustic waves emitted by the biological sample in response to the ultraviolet light, said at least one transducer positioned such that said at least one transducer and said focusing assembly are coaxial; and
a processor configured to record and process the received photoacoustic waves.

2. The ultraviolet photoacoustic imaging apparatus according to claim 1, wherein the ultraviolet light comprises a wavelength of from about 210 nm to about 320 nm.

3. The ultraviolet photoacoustic imaging apparatus according to claim 1, wherein said focusing assembly comprises an optical assembly of lenses and mirrors configured to focus the ultraviolet light on the biological sample so that a focal point of said focusing assembly coincides with a focal point of said at least one transducer.

4. The ultraviolet photoacoustic imaging apparatus according to claim 1, wherein said focusing assembly is positioned on an XYZ translation stage to perform raster scanning along a surface of the biological sample with simultaneous adjustment of an axial position of said apparatus to compensate for a curvature of the surface of the biological sample.

5. The ultraviolet photoacoustic imaging apparatus according to claim 1, wherein said focusing assembly comprises an optical microscope objective configured to focus the ultraviolet light into a nearly diffraction-limited point.

6. The ultraviolet photoacoustic imaging apparatus according to claim 1, wherein said focusing assembly comprises an oscillating mirror configured to scan an optical focus of a larger focal area as compared to a focal area of said at least one transducer.

7. The ultraviolet photoacoustic imaging apparatus according to claim 1, wherein the ultraviolet light is selected from the group consisting of intensity modulated ultraviolet light and pulsed ultraviolet light.

8. A method for imaging a cell nucleus in a biological sample, the method comprising:
exposing the biological sample to ultraviolet light, wherein the ultraviolet light is focused on an area of the biological sample using a focusing assembly;
transforming optical energy absorbed by the biological sample in response to the ultraviolet light into a photoacoustic wave;
detecting the photoacoustic wave using at least one transducer positioned such that the at least one transducer and the focusing assembly are coaxial and confocal; and
creating an image of the area of the biological sample using a processor, the image being based on a signal generated by the at least one transducer and being representative of the photoacoustic wave.

9. The method of claim 8, wherein the ultraviolet light comprises a wavelength of from about 210 nm to about 320 nm.

10. The method of claim 8, wherein the biological sample is ex vivo.

11. The method of claim 8, wherein the biological sample is in vitro.

12. The method of claim 8, further comprising labeling the biological sample.

13. The method of claim 8, wherein creating an image comprises recording and digitizing the detected photoacoustic wave.

14. The method of claim 13, wherein the image is representative of at least one nucleus in the biological sample.

15. A method for imaging an area in a biological sample in vivo, the method comprising:
exposing an area of the biological sample to ultraviolet light, wherein the ultraviolet light is focused on the biological sample using a focusing assembly;
transforming optical energy absorbed by the area of the biological sample in response to the ultraviolet light into a photoacoustic wave;
detecting the photoacoustic wave using at least one transducer positioned such that the at least one transducer and the focusing assembly are coaxial; and
creating an image of the area of the biological sample using a processor, the image being based on a signal generated by the at least one transducer and being representative of the photoacoustic wave.

16. The method of claim 15, wherein the ultraviolet light comprises a wavelength of from about 210 nm to about 320 nm.

17. The method of claim 15, wherein creating an image comprises recording and digitizing the detected photoacoustic wave by the processor.

18. The method of claim 15, wherein the biological sample comprises a eukaryotic organism.

19. The method of claim 18, wherein the eukaryotic organism is selected from the group consisting of a vertebrate, an invertebrate, a plant, a fungus, and a single-cellular organism.

20. The method of claim 15, wherein the area of the biological sample comprises a cell nucleus.
